(19) United States
(12) Patent Application Publication
(10) Pub. No.: US 2022/0260542 A1
(43) Pub. Date: Aug. 18, 2022

(54) REAL-TIME FOUL SMELL TRACKING SYSTEM USING ULTRALIGHT FLIGHT DEVICE

(71) Applicant: TAESUNG ENVIRONMENTAL RESEARCH INSTITUTE CO., LTD., Ulsan (KR)

(72) Inventors: Gi Yeol YUN, Ulsan (KR); Seok Man KIM, Ulsan (KR)

(21) Appl. No.: 17/618,148

(22) PCT Filed: Dec. 9, 2019
(86) PCT No.: PCT/KR2019/017294

§ 371 Date: Dec. 10, 2021

(30) Foreign Application Priority Data
Oct. 31, 2019 (KR) 10-2019-0137075

(51) Int. Cl. G01N 33/00; G01N 1/22; B64C 39/02; B64D 47/00

(52) U.S. Cl. CPC G01N 33/0062; G01N 1/2273; B64C 2201/12; B64D 47/00; B64C 2201/146; B64C 39/024

(57) ABSTRACT

Provided according to one embodiment of the present disclosure is a real-time odor tracking system using an ultralight flight device, the system comprising: an ultralight flight device which measures odor information while moving in the air; and a server which analyzes and manages information on odor generated from a specific point, on the basis of the odor information collected from the ultralight flight device.

### TECHNICAL FIELD

The present invention relates to a system for real-time odor tracking using an ultralight flight device. More specifically, the present invention relates to a system for analyzing and managing odor information generated from a specific point on the basis of odor information collected from the ultralight flight device.

### BACKGROUND ART

As the industry develops, the influence of odor generated from industrial complexes on the surrounding areas is becoming a social issue. Accordingly, the government enacted the odor prevention act and has legally controlled the amount of generated odor since 2005.

The diffusion degree of odor generated from pollution sources is determined by a terrain or an atmospheric condition, etc. When odor is generated from a specific point, in order to accurately track odor generating sources which affect the odor generation, it is necessary to obtain accurate information on atmospheric conditions, etc., at the time of odor generation. The atmospheric conditions can be measured if enough atmospheric measuring networks are set up. In addition, in order to backtrack the odor generating sources, it is necessary to obtain information on main pollutants produced from the odor generating sources, most of which has been secured by inspecting the process of the odor generating sources, etc.

In this situation, the most important information for the backtracking of the odor generating sources is component analysis of pollutants included when the odor is generated. For accurate component analysis, it is necessary to collect the gas at the time of odor generation in real time.

However, now, odor handling employees irregularly visit the area where civil complaints about odor generation often arise, carrying a simple portable device for collecting the air to collect the air manually. Odor tends to instantaneously appear and disappear due to atmospheric conditions, etc., which makes it difficult to collect the gas for accurate analysis.

Furthermore, the degree of sensing odor varies depending on individual's sense of smell, and the diffusion degree of odor is affected by atmospheric conditions, etc. Thus, for effective analysis, it is essential to accurately measure the concentration of odor and collect in real time the gas at the time of odor generation at the site upon odor generation.

However, the gas collecting at the site at the moment of initial stage of odor management depends on humans. That is, since odor managers visit the site and collect the gas on their own, they fail to collect the gas at the exact time of odor generation due to space/time constraints, resulting in inaccurate odor analysis, etc. As such, there are many problems in odor management.

### SUMMARY OF INVENTION

### Technical Task

The present invention is to solve the above-described problems of the prior art. It is an object of the present invention to provide a system for analyzing and managing information on odor generated from a specific point on the basis of smell information collected from an ultralight flight device.

The object of the present invention is not limited to the aforementioned objects, and other objects that are not mentioned can be clearly understood from the following description.

### Means for Solving the Task

According to an embodiment of the present invention, provided is a system for real-time odor tracking using an ultralight flight device, comprising an ultralight flight device for measuring odor information while moving in the air; and a server for analyzing and managing odor information generated from a specific point on the basis of the odor information collected from the ultralight flight device.

The ultralight flight device may include an unmanned multicopter among unmanned powered flight devices specified in Article 5 (Criteria for ultralight flight devices) of Enforcement Rule of Aviation Safety Act, which is hereinafter referred to as an ultralight flight device.

The ultralight flight device is a multi-rotor shaped platform driven by multiple propellers, and may be classified into versions of Quad (4), Hexa (6), and Octo-Quad (8) Rotors according to missions.

The ultralight flight device may sense odor through the smell information.
When odor is sensed through the smell information, the ultralight flight device may collect the sensed odor.

The ultralight flight device may measure atmospheric environment information.

When odor is sensed through the smell information, the ultralight flight device may track the sensed odor.

In order to collect or track the sensed odor, the ultralight flight device may set a flight path through a ground control system (GCS).

According to an embodiment of the present invention, an integrated monitoring system for real-time odor tracking may comprise a fixed odor measuring device for measuring odor information while being fixed at a specific point; a mobile odor measuring device for measuring odor information while moving on the ground; a drone for measuring odor information while moving in the air; and a server for analyzing and managing information on odor generated from a specific point by obtaining the odor information from the fixed odor sensing device, the mobile odor sensing device and the drone, and processing the odor information obtained from the fixed odor measuring device, the mobile odor measuring device and the drone in different ways.

Also, at least one of the fixed odor measuring device, the mobile odor measuring device and the drone may sense an odor causing substance in real time and transmit the odor information to the server as sensing the odor causing substance, and the server may determine an update frequency of updating the odor information on the basis of a location of the specific point and the odor information.

Also, the server may convert and compute at least one of a smell type, a smell intensity, a complex odor and an odor causing substance concentration using the odor information, linearly increase the update frequency as the smell intensity, the complex odor and the odor causing substance concentration increase, and stepwise increase the update frequency according to a changing rate of the odor information at the specific point on the basis of weather conditions and surrounding odor generation conditions.

Also, the system may further comprise a weather measuring device for measuring weather information, and the server may compare weather information obtained from the weather measuring device and the odor information to analyze an odor generation pattern.

Also, the server may predict odor generation from the odor generation pattern and provide the odor information.

Also, the server may send a notification message to a manager terminal when deciding that odor is generated as a result of analysis of the odor information.

Also, the fixed odor measuring device may comprise an OMS, wherein the OMS may comprise a plurality of sensors arranged in two dimension, learn two-dimensional patterns shown by the plurality of sensors which respond according to types of odor, and determine a type and concentration of a substance included in the odor according to the two-dimensional patterns shown by the plurality of sensors.

### Effect of Invention

According to an embodiment of the present invention, a way of reducing odor can be easily established by measuring or collecting an odor substance generated from a specific point in real time with an odor measuring device and an odor collecting equipment for analysis, and identifying an odor causing substance.

The effects of the present invention are not limited to the above-mentioned effects, and it should be understood that the effects of the present invention include all effects that could be inferred from the configuration of the invention described in the detailed description of the invention or the appended claims.

### BRIEF DESCRIPTION OF DRAWINGS

FIG. 1 is view illustrating an integrated monitoring system for odor tracking according to an embodiment of the present invention;

FIG. 2 is a view illustrating a system block diagram of the integrated monitoring system for odor tracking according to an embodiment of the present invention;

FIG. 3 is a view illustrating a network block diagram of the integrated monitoring system for odor tracking according to an embodiment of the present invention;

FIG. 4 is a view illustrating a flow of collecting odor data according to an embodiment of the present invention;

FIG. 5 is a block diagram illustrating a constitution of an ultralight flight device according to an embodiment of the present invention;

FIG. 6 is a view illustrating an ultralight flight device for monitoring atmospheric environment according to an embodiment of the present invention;

FIG. 7 is a view illustrating an ultralight flight device for collecting odor according to an embodiment of the present invention;

FIG. 8 is a view illustrating a connection of base materials in a collecting module according to an embodiment of the present invention;

FIG. 9 is a view illustrating an arrangement of base materials in the collecting module according to an embodiment of the present invention;

FIG. 10 is a view illustrating a connection of a sensor sensing unit according to an embodiment of the present invention;

FIG. 11 is a view illustrating an example of obtaining odor related data using big data and an odor monitoring system (OMS) according to an embodiment of the present invention; and

FIG. 12 is a view illustrating an example of an OMS according to an embodiment analyzing odor.

### DETAILED MEANS FOR CARRYING OUT THE INVENTION

Hereinafter, the present invention will be explained with reference to the accompanying drawings. The present invention, however, may be modified in various different ways, and should not be construed as limited to the embodiments set forth herein. Also, in order to clearly explain the present invention in the drawings, portions that are not related to the present invention are omitted, and like reference numerals are used to refer to like elements throughout the specification.

Hereinafter, embodiments of the present invention will be explained in more detail with reference to the accompanying drawings.

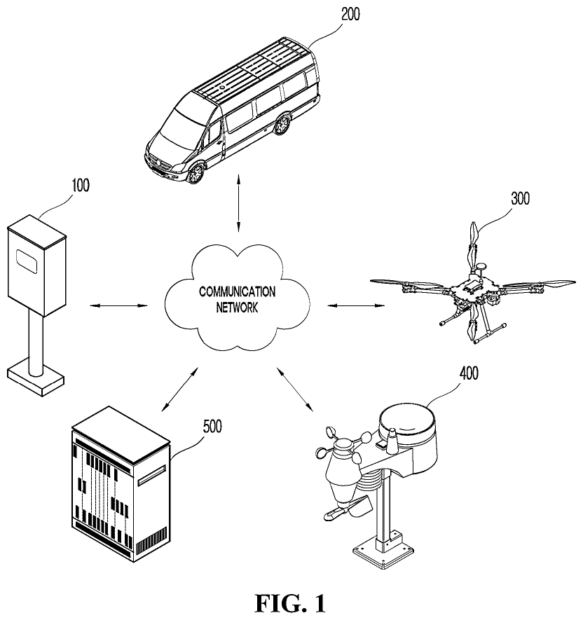

FIG. 1 is a view illustrating an integrated monitoring system for odor tracking according to an embodiment of the present invention.

Referring to FIG. 1, the integrated monitoring system for odor tracking may comprise a fixed odor measuring device 100, a mobile odor measuring device 200, an ultralight flight device 300, a weather measuring device 400, and a server 500, which can communication with each other bidirectionally through a communication network. If the weight of a body of the ultralight flight device 300 is 12 kg or above, a person with a license for the ultralight flight device should directly operate the device or carry a controller for an emergency control.

First, the communication network may include various communication networks, such as radio frequency (RF), a local area network (LAN), a metropolitan area network (MAN), a wide area network (WAN), a mobile communication network, etc., regardless of communication aspects such as wired and wireless communications, etc.

The fixed odor measuring device 100 may measure smell information while being fixed at a specific point and collect the measured smell information.

The mobile odor measuring device 200 may measure smell information while moving on the ground and collect the measured smell information.

The ultralight flight device 300 may measure smell information while moving in the air and collect the measured odor information.

Each of the fixed odor measuring device 100, mobile odor measuring device 200 and the ultralight flight device 300 may sense an odor causing substance in real time and transmit smell information to the server 500 when sensing an odor causing substance.

The weather measuring device 400 may measure and collect weather information.
The server 500 may receive smell information collected from the fixed odor measuring device 100, mobile odor measuring device 200, ultralight flight device 300, etc., and analyze and manage information on odor generated from a specific point on the basis of the odor information collected from various devices.

The server 500 may convert and compute at least one of a smell type, a smell intensity, a complex odor and an odor causing substance concentration using the odor information.

The server 500 may receive weather information collected from the weather measuring device 400, and compare the weather information and odor information to analyze a pattern of odor generation.

The server 500 may predict odor generation from the pattern of odor generation and provide predicted odor information according to the result of prediction of odor generation.

The server 500 may send an alert notification message of odor generation to a manager terminal (not illustrated) when deciding that odor is generated as a result of analysis of odor information.

According to an embodiment, the integrated monitoring system for odor tracking measures a smell type, a smell intensity, a complex odor and an odor causing substance concentration in real time, and may accordingly enable quick preparation of a measure in response to civil complaints when civil complaints about odor arise.

The integrated monitoring system for odor tracking may be classified into the fixed odor measuring device 100 for sensing odor causing substances in real time and transmitting information to the server 500 and the server 500 for receiving the information and displaying the same.

The integrated monitoring system for odor tracking may in real time store in database measurement data of an odor sensor which is measured at the site.

The integrated monitoring system for odor tracking may be classified into odor measuring devices such as the fixed odor measuring device 100, mobile odor measuring device 200 and ultralight flight device 300, a weather measuring device such as the weather measuring device 400, and the server 500. Data transmission between the odor measuring devices and the server 500 may be carried out wirelessly. An odor measurement result measured at a point where the odor measuring device is positioned may be transmitted to the server 500 to be displayed.

The fixed odor measuring device 100, mobile odor measuring device 200 or ultralight flight device 300 may transmit the measured odor measurement result to the server 500. At this time, a transmission frequency of transmitting the odor measurement result to the server 500 may be determined differently depending on situations. The transmission frequency may vary according to the odor measurement result and odor measurement position. For example, the transmission frequency may be determined based on how high a smell intensity, concentration or dilution factor is according to the odor measurement result. As the smell intensity, concentration or dilution factor increases, the transmission frequency may stepwise increase. As another example, the transmission frequency may be high when a change of a predetermined value or more is expected at the current odor measurement position in a predetermined time (for example, in real time). For example, the transmission frequency may be high when a dramatic change in the odor measurement result is expected at the current odor measurement position based on weather conditions such as wind, etc., and surrounding odor generation conditions. The transmission frequency may be determined according to a size of the expected change.

The server 500 may determine the location of odor generation by using odor information received from the fixed odor measuring device 100, mobile odor measuring device 200 and ultralight flight device 300, and weather information received from the weather measuring device 400. The server 500 may process the odor information received from the fixed odor measuring device 100, mobile odor measuring device 200 and ultralight flight device 300 in different ways and use the information, in order to determine the location of odor generation.

For example, the server 500 may grant different reliabilities to odor information received from the fixed odor measuring device 100, mobile odor measuring device 200 and ultralight flight device 300. The reliabilities of hardware for odor measurement mounted on the fixed odor measuring device 100 and mobile odor measuring device 200 may be higher than the reliability of hardware for odor measurement mounted on the ultralight flight device 300. As such, the server 500 may perform integrated monitoring for odor tracking by granting a high weight value to the odor information received from the fixed odor measuring device 100 and mobile odor measuring device 200 and granting a low weight value to the ultralight flight device 300.

As another example, the server 500 may perform odor monitoring by reflecting characteristics of hardware for odor measurement included in the fixed odor measuring device 100, mobile odor measuring device 200 and ultralight flight device 300. As an example, when hardware for odor measurement with high reliability in monitoring hydrogen sulfide is mounted on the fixed odor measuring device 100, hardware for odor measurement with high reliability in monitoring ammonia is mounted on the mobile odor measuring device 200, and hardware for odor measurement with high reliability in monitoring complex odor is mounted on the ultralight flight device 300, the server 500 may perform integrated monitoring for odor tracking (for example, determining the location of odor generation) by granting a highest weight value to the odor information obtained from the fixed odor measuring device 100 when performing the monitoring of hydrogen sulfide, granting a highest weight value to the odor information obtained from the mobile odor measuring device 200 when performing the monitoring of ammonia, and granting a highest weight value to the odor information obtained from the ultralight flight device 300 when performing the monitoring of complex odor.

As another example, the server 500 may apply time difference to odor information received from the ultralight flight device 300 when performing integrated monitoring for odor tracking. When an altitude which is a reference altitude when performing monitoring of odor is close to the ground, time difference may exist in order for odor information measured at a position of high altitude to be reflected in a position of low altitude. Accordingly, the server 500 may use weather information received from the weather measuring device 400 to determine whether the air current at the position of the ultralight flight device 300 is an ascending air current or a descending air current, and determine an intensity of the air current. The server 500 according to an embodiment may reflect odor information received from the ultralight flight device 300 at a rate lower than the predetermined rate (for example, 5%), when the air current at the position of the ultralight flight device 300 is an ascending air current. Or, the server 500 according to an embodiment may perform monitoring of odor on the ground by reflecting odor information received from the ultralight flight device 300 at a time interval which is inversely proportional to the intensity of the air current, when the air current at the position of the ultralight flight device 300 is a descending air current.

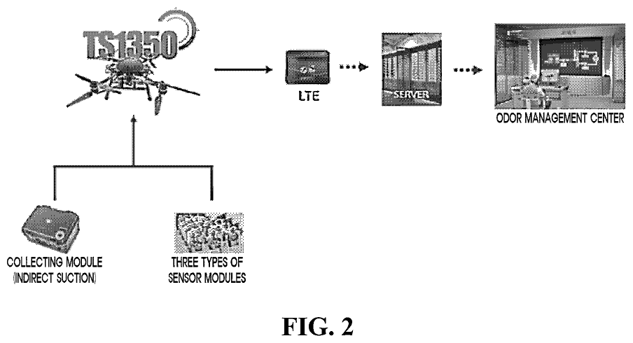

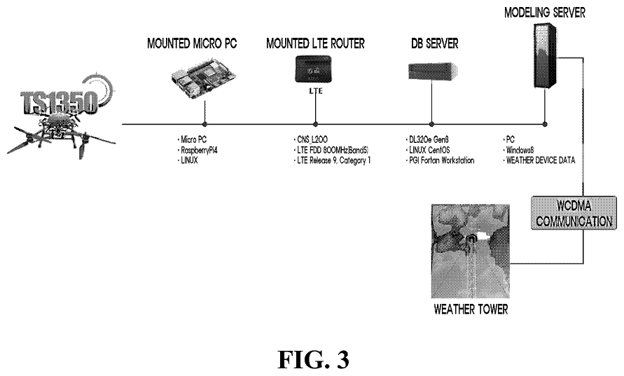

FIG. 2 is a view illustrating a system block diagram of the integrated monitoring system for odor tracking according to an embodiment of the present invention, and FIG. 3 is a view illustrating a network block diagram of the integrated monitoring system for odor tracking according to an embodiment of the present invention.

As illustrated in FIG. 2 and FIG. 3, the integrated monitoring system for odor tracking may analyze and manage data on surrounding odor by measuring in real time main odor causing substances (for example, complex odor, hydrogen sulfide, ammonia, TVOCs, etc.) generated from a specific point or national industrial complexes in which odor emitting companies are concentrated and weather information (wind direction, wind speed, temperature, humidity, etc.), and transmitting collected data (smell intensity, concentration, diffusion path, weather information, etc.) to a control system implemented into the server 500 remotely, using a wireless communication network (WCDMA, LTE, etc.).

The integrated monitoring system for odor tracking may configure an unmanned odor collecting device as an integral type and a separate type according to consumer's demands, automatically collect a sample in steps when exceeding an odor reference value, and provide a function allowing a manager to remotely collect odor from the site at any time.

The integrated monitoring system for odor tracking may automatically send a text message of alert and state to a manager using SMS and APP when odor is generated and odor of a threshold value or more is generated.

As for the integrated monitoring system for odor tracking, an unmanned odor collecting system and a weather measuring system may be manufactured as an integral type and a separate type according to options.

The integrated monitoring system for odor tracking includes an odor sensing device and an information processing system. The weather measuring device 400 may analyze the generation pattern by collecting weather information and comparing the information with odor information, and may be implemented into an integrated odor information management system enabling preparation of a measure of predicting and preventing odor generation by displaying the sensed and measured odor information outside in real time or periodically.

The integrated monitoring system for odor tracking may provide total condition services regarding odor, monitor fine dust in real time using smartphone applications and PC, confirm the surrounding fine dust level by interconnecting with CCTV, electronic display, etc., and enable an immedate response upon event occurrence through prediction and alert notification.

As a method for collecting odor data, odor collected from an odor causing source and weather data are transmitted to a signal converter, and the odor and weather signal converter may convert the collected analog signal to a digital signal, and process a physical signal with the smell type, smell intensity and concentration to transmit the signal to a data analyzer.

The odor data analyzer may process the data collected from the signal convert in various forms and store the same in a storing device in the analyzer.

The analysis data of the odor measuring device may include real-time data, odor intensity data, odor diffusion three-dimensional data, etc.

The odor intensity data is data measured by an automatic odor measuring device on smell intensity, smell type, concentration and dilution factor for each gas with respect to measurement ranges and odor intensities, and as for the odor intensity data, measurement data may be stored in order to send an alert text message and display odor modeling when odor of a threshold value or more is generated.

The odor diffusion three-dimensional data is three-dimensional data made by the server 500 through a modeling program by processing actual odor into a signal when odor of a predetermined value or more is generated, and then storing the signal as a file, and when a file of the measured odor information is created, abnormal odor data is stored in a management program, and the created file may be stored along with data on smell intensity, smell type, concentration, dilution factor, etc.

As a method for analyzing odor data, the odor data may be processed and analyzed by odor data processing S/W of the odor analyzer on the basis of odor data collected from the odor measuring device by the signal converter.

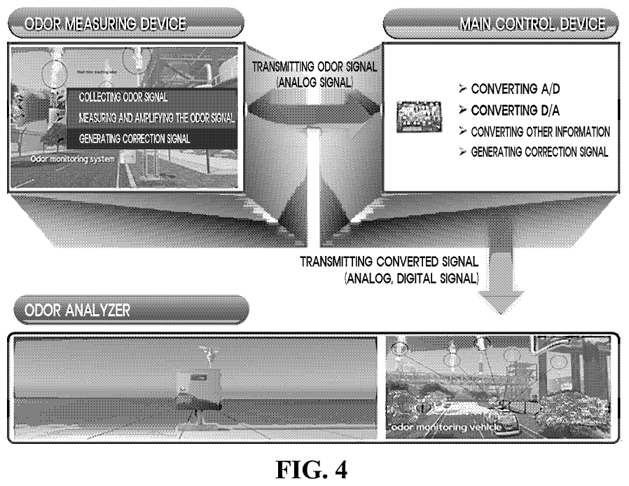

FIG. 4 is a view illustrating a flow of collecting odor data according to an embodiment of the present invention.

As illustrated in FIG. 4, the odor measuring device may collect an odor signal, perform measurement and amplification of the odor signal, generate a correction signal, and transmit the odor signal as an analog signal.

The main control device may perform a process of A/D conversion, D/A conversion, other information conversion, correction signal generation, etc. on the odor signal, and transmit a signal converted into an analog signal or a digital signal to the odor analyzer.

When determined as HALT (a hardware failure or failure in odor measuring device in a state in which the system cannot receive data at all), response failure (a communication failure state due to network disconnection), the main control device may wait for 10 seconds, and it may be processed as time-out after 10 seconds.

As for the measurement data transmitted in real time from the automatic odor measuring device and the measurement data returned by a request of a communication server, an end of transmission (EOT) signal is transmitted to notify a management system communication server of completion of transmission when transmission is terminated.

The transmission and reception data is filled from the right side of the number of digits of a format defined by the communication protocol, and when no data is present or the data is a fixed number of digits or less, a blank value may be filled therein.

The transmission side transmits the last data and receives an EOT signal from the reception side, and then transmission is terminated. Upon completion of transmission, the connection may be closed.

As a way of transmitting odor data, TCP/IP is used for transmission and reception with a management center. When the automatic odor measuring device transmits data to the management center, the management center may be the server 500. When the management center transmits a telecommand to the odor measuring device, the odor measuring device may be the server 500.

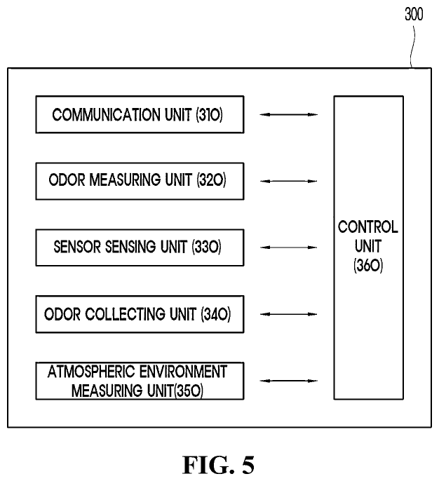

FIG. 5 is a block diagram illustrating a constitution of an ultralight flight device 300 according to an embodiment of the present invention.

Referring to FIG. 5, the ultralight flight device 300 may comprise a communication unit 310, an odor measuring unit 320, an odor sensing unit 330, an odor collecting unit 340, an atmospheric environment measuring unit 350, and a control unit 360.

First, the communication unit 310 may perform a communication function of transmitting and receiving information in communication with an external device, and for example, transmit the measured odor information to the server 500.

The odor measuring unit 320 may measure odor information, and measure and collect surrounding odor information that changes in real-time or periodically while the ultralight flight device 300 moves in the air.

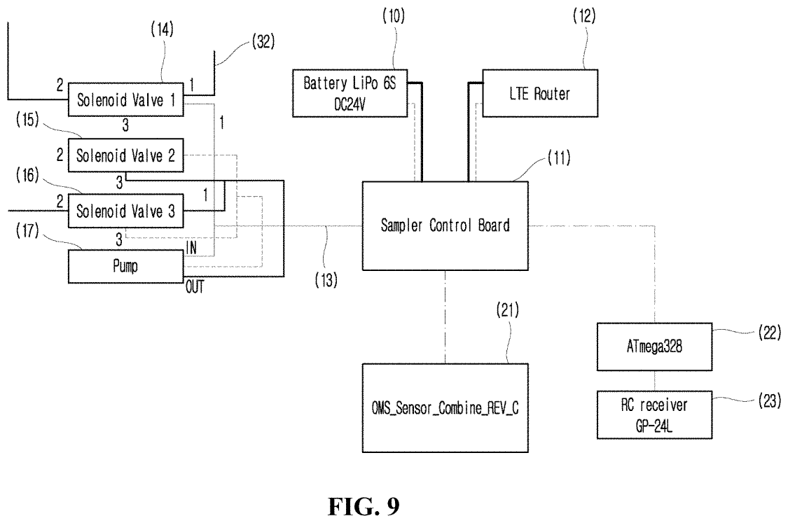

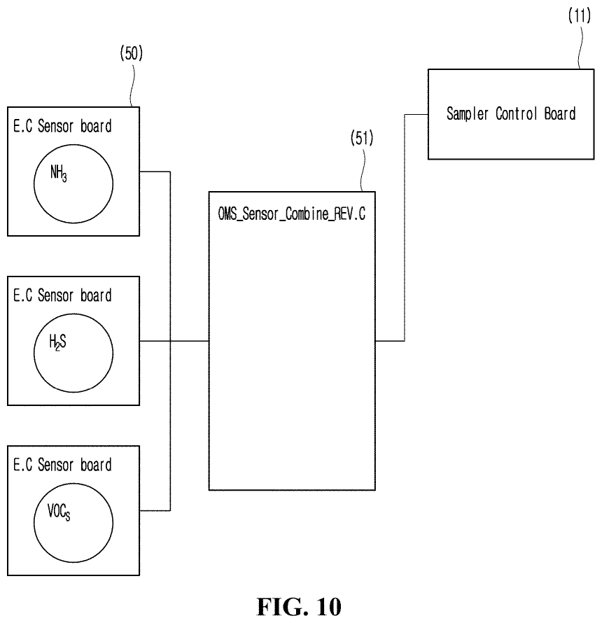

The sensor sensing unit 330 may sense odor through measurement information, and for example, confirm whether the odor is sensed at a point where smell is measured on the basis of odor information. The sensor sensing unit 330 comprises PID (VOCs), E.C (H2S, NH3) sensors, and a result value is displayed only by a voltage up to a sensor integrated board 51. The voltage value at this time is designed to output a voltage up to 5 v during Full Scale. When the voltage is delivered through wiring from a sensor board 50 to the sensor integrated board 51, SPI communication is performed in the sensor integrated board 51 with a STM32 microprocessor chip inside a control board 11 (FIG. 9) (see FIG. 10). When transmitted from the control board to the odor monitoring system, data is transmitted and received through an LTE router 12 (FIG. 9) (see FIG. 2).

The odor collecting unit 340 may collect sensed odor when odor is sensed through a gas sensor. The odor collecting unit 340 relates to a sample collecting device mounted on an ultralight flight device for collecting an odor causing substance included in the air in a gaseous state.

The odor collecting unit 340 may be mounted on the ultralight flight device 300 to collect samples of the odor causing source in the air through the flight. The odor collecting unit 340 is a method using the principle of a lung sampler. The method sucks in the air inside a box with a vacuum pump in order for the box to be in a vacuum state so that an external gaseous sample can slowly flow into a Tedlar bag, which complies with ES01115 of a standard test method for air pollution (sampling methods in ambient atmosphere). The control board which receives a collection command from the RF signal or odor monitoring system operates/stops a solenoid valve or a vacuum pump through a PWM GPIO port. The detailed operation order therefor is shown in FIG. 9. The available voltage is pulled in the control board 11 of the collecting module using the power of a battery 10 mounted on the ultralight flight device 300 and is outputted to the LTE router 12, and power 13 for operating the pump and solenoid valve is pulled in.

The SPI communication is possible in the control board 11 and sensor integrated board 21. The control board 11 and an ATmega 22 connect GPIO 5 PIN to communicate with 1-standby, 2-start, 3-collecting, 4-collection completed, 5-reset, etc. The ATmega 22 is connected to an RC receiver 23 to transmit and receive a PWM signal. The LTE router 12 may receive the collection command from the system for odor monitoring and receive the collection command from a pilot who operates the ultralight flight device 300. Therefore, when the air is pulled in a vacuum box of the collecting module through a second valve of S/V_1 (14) by the signal received from the LTE router 12 or RC receiver 23, the air inside the vacuum box is pulled in a pump 17 through a first valve of S/V_2 (15) and a third valve of S/V_3 (16), and the air is connected with a third valve of S/V_2 (15) and a first valve of S/V_3 (16) in the pump 17, it is emitted through a second valve of S/V_3 (16).

The atmospheric environment measuring unit 350 may measure atmospheric environment information, and measure and collect surrounding atmospheric environment information that changes in real-time or periodically while the ultralight flight device 300 moves in the air. The control unit 360 may control the operations of the communication unit 310, odor measuring unit 320, sensor sensing unit 330, odor collecting unit 340 and atmospheric environment measuring unit 350 to be normally performed. The control unit 360 may control to track the sensed odor when odor is sensed through the gas sensor and set a flight path of the ultralight flight device 300 in order to track the sensed odor.

When odor is sensed through the gas sensor, the control unit 360 may be controlled to collect the sensed odor. In order to collect the sensed odor, the flight path of the ultralight flight device 300 may be set. The weather information obtained from the weather measuring device 400 may include information indicating overall weather conditions of a broad area, and atmospheric environment information obtained from the atmospheric environment measuring unit 350 may include information more precisely indicating surrounding situations of the ultralight flight device 300. The flight path of the ultralight flight device 300 may be determined depending on weather information, atmospheric environment information (e.g., wind direction), distribution status of odor, etc. As an example, the control unit 360 may determine the flight path of the ultralight flight device 300 on the basis of the wind direction, current temperature, distribution status of odor, surrounding terrain and conditions of surrounding facilities. For example, the control unit 360 may determine the flight path of the ultralight flight device 300 to fly in reverse direction of the wind direction when the odor intensity currently collected is a threshold value or above, and to fly in the same direction as the wind when the odor intensity currently collected is less than a threshold value. As another example, the control unit 360 may determine the flight path of the ultralight flight device 300 to fly at an altitude higher than the predetermined height when an ascending air current is generated, and to fly at an altitude lower than the predetermined height when a descending air current is generated. As another example, when there is a mountain range, the control unit 360 may determine the flight path of the ultralight flight device 300 to fly in parallel with the mountain range. Additionally, in this case, when the height of the mountain range is a threshold value or above, the control unit 360 may determine the flight path of the ultralight flight device 300 to fly at an altitude lower than the height of the mountain range. When the altitude of the mountain range is high, since the odor cannot go over the mountain range, more odor information may be obtained when the ultralight flight device 300 flies in parallel with the mountain range at an altitude lower than the height of the mountain range. As another example, the control unit 360 may determine surrounding situations by granting different weight values or time differences in the weather information obtained from the weather measuring device 400 and atmospheric environment information obtained from the atmospheric environment measuring unit 350, and accordingly determine the flight path of the ultralight flight device 300. For example, when a first direction, which is a wind direction according to the weather information obtained from the weather measuring device 400, is different from a second direction, which is a wind direction according to the atmospheric environment information obtained from the atmospheric environment measuring unit 350, the wind direction may be determined by granting a higher weight value to the second direction at present, and the first direction after a predetermined time has passed. Also, the control unit 360 may determine the flight path on the basis of the wind direction determined as above. Or, when the first wind direction obtained from the weather measuring device 400 is different from the second wind direction obtained from the ultralight flight device 300, the server 500 may determine the path of the ultralight flight device 300 on the basis of the first wind direction at a first time and determine the path of the ultralight flight device 300 on the basis of the second wind direction at a second time in which a predetermined time has passed after the first time. The atmospheric environment information obtained from the atmospheric environment measuring unit 350 may precisely indicate in real-time the information on the current surrounding situations of the ultralight flight device 300. However, the weather information obtained from the weather measuring device 400 may more indicate overall information on the situations in a much broader area. Therefore, when the weather information is different from the atmospheric environment information, the control unit 360 may determine the flight path according to the atmospheric environment information at first, but determine the flight path by reflecting the weather information with a time difference. For example, the control unit 360 may first determine the flight path to an odor causing source predicted on the basis of the second direction, and after the predetermined time (e.g., 20 second) has passed, update the flight path to an odor causing source predicted on the basis of the first direction. Additionally, when a difference between the weather information and the atmospheric environment information is greater than a predetermined level (e.g., a difference between the first direction and the second direction is 90° or above), the control unit 360 may determine the flight path by granting a higher weight value to the weather information than the atmospheric environment information. The weather measuring device 400 stably obtains weather information by using hardware with relatively high reliability, but the atmospheric environment measuring unit 350 obtains information by using relatively simple hardware. Therefore, when the difference between the weather information and atmospheric environment information is greater than the predetermined level, the control unit 360 may determine the flight path by granting a higher weight value to the weather information than the atmospheric environment information. Additionally, the degree of granting a higher weight value to the weather information than the atmospheric environment information may be determined depending on the degree of difference between the weather information and atmospheric environment information. For example, as the difference between the weather information and atmospheric environment information becomes greater, the weather information may be granted a higher weight value than the atmospheric environment information. For example, when the difference between the first direction and the second direction is over 150°, the weight value applied to the second direction may be 0 (the second direction is ignored).

According to an embodiment, the ultralight flight device 300 is implemented into one device, and thus may perform both atmospheric environment monitoring function and odor collecting function. In addition, the ultralight flight device 300 may be distinguished as separate devices such as an ultralight flight device for monitoring atmospheric environment and an ultralight flight device for collecting odor.

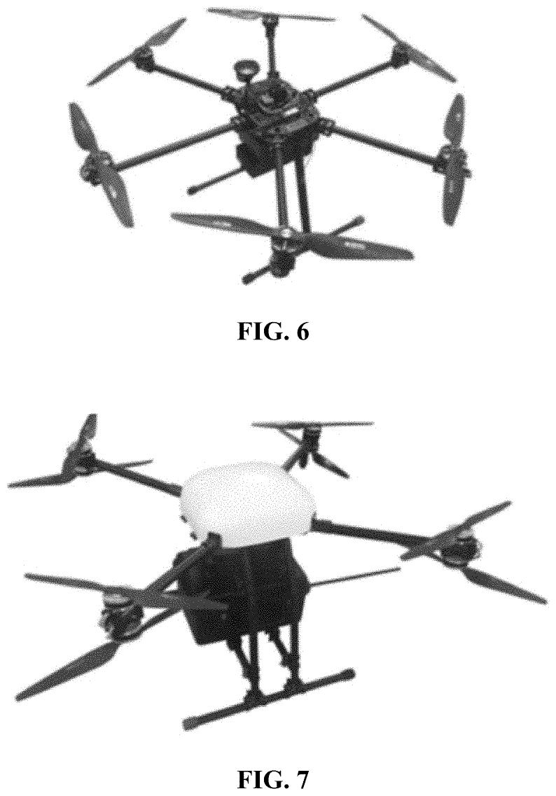

FIG. 6 is a view illustrating an ultralight flight device for monitoring atmospheric environment according to an embodiment of the present invention, and FIG. 7 is a view illustrating an ultralight flight device for collecting odor according to an embodiment of the present invention. The ultralight flight device for monitoring atmospheric environment as illustrated in FIG. 6, and the ultralight flight device for collecting odor as illustrated in FIG. 7 may be distinguished as separated devices, and used in the real-time system for odor tracking using the ultralight flight device 300.

For example, the ultralight flight device 300 may be classified into an ultralight flight device for collecting odor, an ultralight flight device for sensing odor, and an ultralight flight device for monitoring environment according to function, but is not limited thereto, and may be operated as one ultralight flight device performing all functions.

The function of measuring and collecting the atmospheric environment information in real time by using the ultralight flight device 300 in which the odor gas sensor, fine dust sensor, etc. are mounted, transmitting the information to a ground control room implemented as the server 500, and tracking the odor causing source in connection with diffusion modeling, may be provided.

An indirect suction manner prescribed by the process test standards of air pollutants may be used by mounting the collecting device on the ultralight flight device 300, and it may become easy to collect the odor in high and dangerous places such as factory chimneys.

If the flight path is set in advance in an operation program of the ultralight flight device 300 for the flight of tracking, sensing and collecting odor using the ultralight flight device 300, a function of performing a mission using the ultralight flight device 300 at a desired location may be provided.

As such, according to an embodiment of the present invention, a way of reducing odor may be easily established by measuring or collecting the odor substance generated at a specific point with the real-time odor measuring device and odor collecting equipment for the analysis, and identifying the odor causing substance.

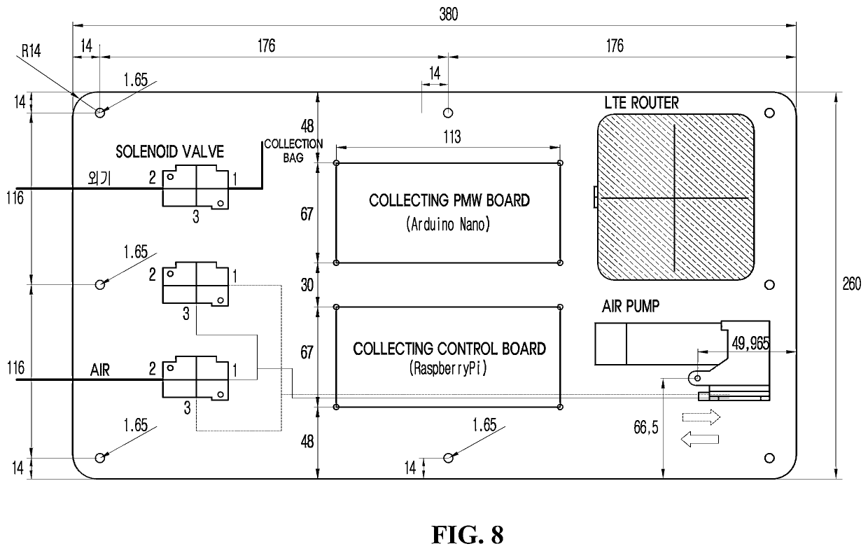

FIG. 8 is a view illustrating a connection of base materials in a collecting module according to an embodiment of the present invention. As illustrated in FIG. 8, the collecting module may collect the air including odor that flows in from the outside, and include a collecting PWM board and a collecting control board.

FIG. 9 is a view illustrating an arrangement of base materials in the collection module according to an embodiment of the present invention. The odor collecting module may include a plurality of base materials, and include a control board 11.

FIG. 10 is a view illustrating a connection of a sensor sensing unit according to an embodiment of the present invention. The sensor sensing unit may include a plurality of sensors, and the odor sensor sensing unit may analyze odor information according to the reaction of the plurality of sensors. According to an embodiment, the sensor sensing unit may be included in the OMS.

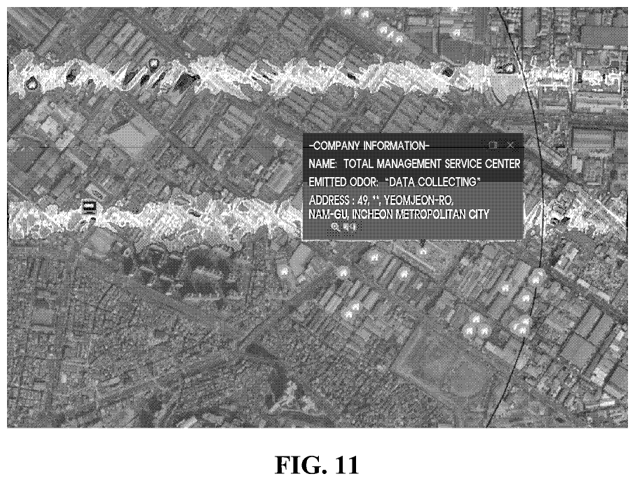

FIG. 11 is a view illustrating an example of obtaining odor related data using big data and an odor monitoring system (OMS) according to an embodiment of the present invention.

The server 500 according to an embodiment may establish big data. For example, the server 500 may establish big data including all of information on factories involved in odor, weather information, information on odor in the air, measurement information on odor, etc. The information on factories involved in odor may include location information about factories, odor information that factories are expected to emit, time when factories emit odor substances, types of odor substances that were emitted by factories in the past, etc. The server 500 may establish big data including various information related to odor to determine a point which is the cause of odor in real time. For example, the server 500 may use big data to determine an odor causing point that is expected to affect the location where civil complaints about odor are filed when the civil complaints about odor are filed.

The server 500 and/or OMS may classify the types and intensities of smell information employing random forest based machine learning and artificial intelligence, and predict dilution factors of smell information by fusing real-time data and accumulated data (big data).

Regarding random forest based machine learning and artificial intelligence for classifying the types and intensities of smell information, temperature, humidity and sensor data input to learning database may be used as independent variables for model generation. Patterns may be classified into classes on the basis of the types and intensities. The classified class values may be stored and displayed as predictive values. A class value having the highest probability may be stored and displayed as a predictive value by estimating the probability of belonging to each class with dependent variables.

In particular, the smell intensity and dilution factor are consistent with Weber-Fechner's law, and the law may be applied to the model generating and predicting process. The smell intensity may be calculated by a formula such as “a +K*log(dilution factor).”

As such, according to an embodiment of the present invention, a way of reducing odor can be easily established by measuring or collecting an odor substance generated from a specific point with an odor measuring device and an odor collecting equipment in real time for analysis, and identifying an odor causing substance.

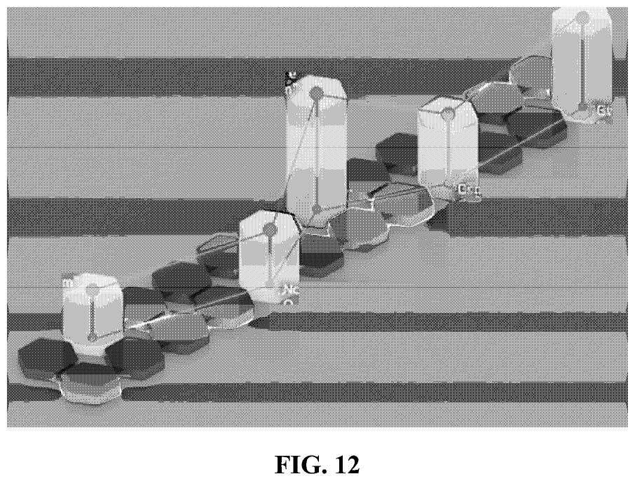

FIG. 12 is a view illustrating an example of an OMS according to an embodiment analyzing odor.

The OMS according to an embodiment may obtain and analyze odor information. For example, the OMS may analyze odor and specifically determine components included in the odor and concentrations of the components, etc. The OMS may comprise a plurality of sensors and analyze odor according to the degree of response of each sensor. For example, the OMS may obtain two-dimensional pattern types shown by the plurality of sensors according to the degree of response of the plurality of sensors arranged in two dimension, and determine causing substances and concentrations of the causing substances according to the obtained two-dimensional pattern types. For example, in the case of garlic smell, methyl acrylate may be 30 ppm, and ethyl acrylate may be 2 ppm. As another example, in the case of suffocating pungent smell, propenylbenzene may be 25 ppm, and NH3 may be 8 ppm.

As such, the OMS may comprise a plurality of sensors arranged in two dimension which show different patterns for each smell, and learn a relationship between the types of odor and the patterns of the plurality of sensors arranged in two dimension. For example, the obtained odor is analyzed using Sift-MS to obtain a result thereof, the OMS learns the analyzed result, and thereby the OMS may analyze odor. In this case, although the OMS is much lighter hardware than the Sift-MS, it may perform accurate odor analysis using the learning result through the Sift-MS.

The above-described description of the present invention is intended for illustration, and a person having ordinary knowledge in the art to which the present invention pertains will understand that the present invention may be easily modified in other specific forms without changing the technical spirit or essential features of the present invention. Therefore, it should be understood that the embodiments described above are exemplary in all respects and not restrictive. For example, each component described as a single type may be implemented in a distributed manner, and similarly, components described as distributed may be implemented in a combined form.

The scope of the present invention is defined by the accompanying claims. It should be construed that all modifications and embodiments derived from the meaning and scope of the claims and their equivalents fall within the scope of the present invention.

Meanwhile, the above-described method can be written as a program that can be executed in a computer, it can be implemented in a general-purpose digital computer to operate the program using a computer-readable recording medium. In addition, the structure of the data used in the above-described method can be recorded on the computer-readable recording medium through various means. The computer-readable recording medium may include a storage medium such as a magnetic storage medium (for example, a ROM, a RAM, a USB, a floppy disk, a hard disk, etc.) and an optical reading medium (for example, a CD-ROM, a DVD, etc.).

A person having ordinary knowledge in the art to which the present embodiment pertains will appreciate that the present invention may be embodied in a modified form without departing from the essential characteristics of the above description. Therefore, the disclosed methods should be considered in descriptive sense and not for purposes of limitation. The scope of the present invention is shown in the claims rather than the foregoing description, and all differences within the scope will be construed as falling within the present invention.

What is claimed is:

1. A system for real-time odor tracking using an ultralight flight device, comprising:
an ultralight flight device for measuring odor information while moving in the air; and
a server for analyzing and managing odor information generated from a specific point on the basis of the odor information collected from the ultralight flight device.

2. The system of claim 1, wherein ultralight flight device senses odor through an odor gas sensor.

3. The system of claim 2, wherein the ultralight flight device collects the sensed odor when the odor is sensed through the gas sensor.

4. The system of claim 2, wherein the ultralight flight device measures atmospheric environment information.

5. The system of claim 2, wherein the ultralight flight device tracks the sensed odor when odor is sensed through the odor information.

6. The system of claim 1, further comprising:
a weather measuring device for obtaining weather information, wherein
when a first wind direction obtained from the weather measuring device is different from a second wind direction obtained from the ultralight flight device, the server determines a path of the ultralight flight device on the basis of the first wind direction at a first time, and determines a path of the ultralight flight device on the basis of the second wind direction at a second time after a predetermined time has passed since the first time.

7. The system of claim 1, wherein when a difference between the first wind direction obtained from the weather measuring device and the second wind direction obtained from the ultralight flight device is a predetermined angle or above, the server determines a path of the ultralight flight device by granting a higher weight value to the first wind direction than the second wind direction.
